(19) United States
(12) Patent Application Publication
(10) Pub. No.: US 2009/0291106 A1
(43) Pub. Date: Nov. 26, 2009

(54) BIODEGRADABLE BLOCK COPOLYMERS WITH MODIFIABLE SURFACE

(75) Inventors: Achim Göpferich, Sinzing (DE); Jörg Tessmar, Regensburg (DE); Michaela Schulz, Regensburg (DE); Torsten Blunk, Pentling (DE); Antonios Mikos, Houston, TX (US)

Correspondence Address: STOUT, UXA, BUYAN & MULLINS LLP, 4 VENTURE, SUITE 300, IRVINE, CA, 92618, US

(73) Assignee: MAST BIOSURGERY AG, Zurich (CH)

(21) Appl. No.: 12/505,449

(22) Filed: Jul. 17, 2009

Related U.S. Application Data
(63) Continuation of application No. 10/019,797, filed on Jul. 26, 2002.

(51) Int. Cl. A61K 9/00; C12N 5/06; C07K 1/04; C40B 40/14

(52) U.S. Cl. 424/400; 435/402; 530/345; 506/20

(57) ABSTRACT

A block copolymer containing a) a hydrophobic biodegradable polymer, b) a hydrophilic polymer and c) at least one reactive group for covalent binding of a surface-modifying substance d) to the hydrophilic polymer b) is disclosed. Shaped bodies are formed to consist of the block copolymer and are utilized as carriers for tissue culture and active substances and for controlled release and targeted administration of active substances.

### CROSS-REFERENCE

This application is a continuation of U.S. application Ser. No. 10/019,797, filed Jul. 26, 2002, the entire contents of which are hereby incorporated by reference.

### BACKGROUND OF THE INVENTION

1. Field of the Invention

The disclosure relates to block copolymers with a hydrophobic biodegradable component and a hydrophilic biocompatible component, which permit the selective binding of surface-modifying substances and at the same time can suppress the non-selective adhesion of unwanted substances, and to shaped bodies produced therefrom.

Such block copolymers are particularly suitable as carriers for cells for tissue culture, as carriers for active substances such as medications, in particular for controlled release (drug delivery system) and for targeted administration of active substances (drug targeting).

2. Description of Related Art

Biomaterials, which include the block copolymers according to the disclosure, play a dominant role in a range of medical applications. The term biomaterials relates to substances which assume a specific function in human or animal body as substitute substances for endogenous materials. Examples of this are metals or polymers, such as, for example, those used in total endoprothesis in the region of the hip joint. A disadvantage of many biomaterials, which are only used temporarily in the body, such as pins or plates in the surgical field, for example, is that they have to be removed after application. For this reason, at the beginning of the seventies an intensive search was started for biodegradable materials which degrade into fragments tolerated by the body during the application.

The term “biodegradable” means that the biological system, into which the material is introduced, contributes to its degradation [Vert, M et al “Degradable Polymers and Plastics” Redwood Press Ltd. (1992) 73-92]. Those particularly worthy of note are biodegradable polymer materials which degrade into oligomers or monomers. Surgical suture material or degradable carriers of medicinal agents are mentioned as examples of their application.

The successful application of biodegradable polymers has led to an intensive search for new synthetic materials, from which a plurality of different polymer classes resulted, such as poly(a-hydroxyesters), poly(b-hydroxyesters), polyanhydrides, polycyanoacrylates and many others [Göpferich, A. (1997) 451-472; Göpferich, A.: Biomaterials 17 (1996a) 103-114; Göpferich, A. Eur. J. Pharm. Biopharm. 42 (1996b) 1-11].

A particular characteristic of these polymers is their low solubility in aqueous media, which only improves through polymer chain degradation, i.e. hydrolysis to lower-molecular oligomers or monomers, and thus leads to erosion of these materials.

Besides the development of synthetic biodegradable polymers, an intensive search was instigated at the same time for natural polymers, which have similar properties. Examples of these are collagens, hyaluronic acid, alginate and cellulose derivatives [Park, K. et al: Biodegradable Hydrogels for Drug Delivery (1993)]. With these substances, it is accepted to some extent that they have an increased water solubility. To lower the water solubility, natural polymers are often chemically modified, e.g. by etherification and esterification of functional groups in the polymer chain or by cross-linkage of individual strands. By way of example, the cross-linkage of collagens, gelatine or alginate are mentioned here.

Various biodegradable polymers differ above all by the rate of polymer chain degradation and erosion. This is important for many applications, in which the polymer chain degradation must extend over a defined time period, such as in the case of release of medicinal agents, for example.

It is essential for the medicinal application of synthetic, part-synthetic and natural biodegradable polymers that they are compatible with the biological system into which they are introduced. For applications in human or animal organisms, individual structural elements, such as oligomers or monomers, must not be toxic and the polymers may trigger, at most, a moderate inflammatory reaction in the tissue.

The above-mentioned biodegradable polymers are currently used for the controlled release of medicinal agents (drug delivery) [Göpferich, A. Eur. J. Pharm. Biopharm. 42 (1996b) 1-11] and as carriers for cells (tissue engineering) [Langer, R and Vacanti, J. P. Science 260 (1993) 920-926].

As part of the drug delivery, biodegradable polymers release medicinal agents in a controlled manner by diffusion, erosion, swelling or osmotic effects.

In the field of tissue engineering, biodegradable polymers as used as porous “sponges,” for example, on which cells can adhere, proliferate and be differentiated. While the cells develop to a tissue band, the polymer carrier degrades and a tissue results which may be transplanted into the human or animal body.

Examples of tissues currently produced in this way are, inter alia, cartilage, bone, fatty tissue and vessels.

The application of biodegradable polymers in the fields of tissue engineering and drug delivery set particular requirements for these materials.

Besides the already mentioned biocompatibility of the polymers and their degradation products, these applications set particular requirements for the surface properties of the polymers.

Some examples from the field of drug delivery shall be named firstly:

1. An adsorption of molecules (for example, medicinal agents, proteins and peptides) onto the polymer surfaces is frequently observed. This can result in the biodegradable medicinal agent carrier not releasing its dosage to the desired extent and not with the desired kinetics. In an extreme case, this can also lead to inactivation of the active substance. The adsorption of active substances is therefore undesirable in many cases and must be suppressed.

2. The compatibility of a biodegradable polymer is greatly dependent on its surface properties. Hence, these polymers in the form of particles in the micrometer and nanometre range are recognized by cells of the immune system such as macrophages, for example, after absorption of endogenous proteins, and subsequently phagocytised. It is therefore necessary to examine the surface properties of small particles as parenteral forms of medicines for their successful use.

3. Biodegradable nanoparticles have long been sought to use for the targeted administration of substances to specific tissue (for example, tumors or central nervous system) (drug targeting). It has been found in this case that endogenous proteins which are adsorbed on the particle surfaces are responsible for where these particles are transported. [Juliano, R. L.: Adv. Drug Delivery Rev. 2 (1988) 31-54]. Hitherto it has only been conditionally possible to achieve a targeted adsorption of these proteins onto the particles. Polymers which allow the targeted modification of their surfaces by simple means are therefore advantageous.

The surface properties of biodegradable polymers also play an important role in the field of tissue engineering:

1. The interactions between cells and polymer determine cell growth and cell differentiation. Natural anchorage mechanisms of the cells are responsible for adhesion of the cells to the polymer surfaces. Proteins such as integrins, for example, allow cells to adhere to specific amino acid sequences. The adhesion to biodegradable polymers occurs as a result of proteins from body fluids or cell culture media adsorbing non-specifically to the polymer surfaces and, in turn, the cells themselves adhering to the corresponding amino acid sequences of the proteins. This non-specific adsorption of proteins causes a plurality of different cells to adhere to the surface. This is above all disadvantageous if a specific cell type is to be adhered to the biodegradable polymer. It is therefore desirable to examine the adsorption of proteins and peptides.

2. The amino acid sequences to which cells adhere are often specific for a cell type, i.e. if the surface of a polymer is coated with a cell-specific sequence, then this cell type preferably adheres.

3. The membrane of a cell carries a series of receptors, in which case the behavior of the cell can be influenced via these receptors. Therefore, if corresponding “signal substances” such as hormones, growth factors or cytokines, for example, are located on the surface of polymers, to which the receptors can bind, the behavior of the cells adhering thereto via the receptors may be influenced via these correspondingly coated polymer surfaces.

The above-mentioned examples show the importance of the surface properties of a biodegradable polymer or the importance of the possibility of selective modification of these surfaces for successful application of the polymer. The modification of surface properties of biodegradable polymers has been the aim of intensive research for some years.

The first attempts at producing biodegradable polymers with modifiable surfaces started from incorporating monomers such as lysin, for example, which contain a functional group to which the molecules can adhere, into the polymer chain of poly(a-hydroxyesters), e.g. polylactide, [Barrera, D. A. “Synthesis and Characterization of a Novel Biodegradable Polymer—Poly(lactic acid-co-lysin)” 1993, Massachusetts Institute of Technology, PHD Thesis].

A disadvantage of these polymers is that the functional groups, in this case amino groups, are only accessed in the surface with difficulty. In order to improve this, oligopeptides were adhered to these functional groups in order to facilitate the binding of new chemical bonds.

A disadvantage is that the non-specific adsorption of unwanted proteins and peptides occurs in the polymer obtained.

This led to new developments in order to obtain a more broadly applicable system [Patel, N., Padera, R., Sanders, G. H., Cannizzaro, S. M., Davies, M. C., Langer, R., Roberts, C. J. Tendler, S. J., Williams, P. M. and Shakesheff, K. M. “Spatially controlled Tissue Engineering on Biodegradable Polymer Surfaces.” 25(1), 109-110, 1998. Controlled Release Society, Inc. Proceed. Int'l. Symp. Control. Rel. Bioact. Mater. 1998]. In this case the binding of biotin to the protein avidin which is very specific is utilized. Biotin is anchored on a polymer surface and biotin is also bound to the substance with which the surface is to be coated. In the presence of avidin, which has several binding points for biotin, the targeted adhesion of the biotinyled compound to the surface then results.

An advantage of the process is that patterns may be generated on the polymer surface. This is important for tissue where a structured arrangement of cells is necessary.

However, a disadvantage is that by anchoring avidin, a protein is used which is exogenous and can therefore lead to undesirable reactions. In addition, the substance to be anchored must first be biotinyled, which complicates the process and thus restricts applicability. At the same time, the surface is coated with avidin, which is undesirable for many applications.

Other methods use a further polymer to adhere surface-modifying substances to the surface of the biodegradable polymer. Hence, polyethylene glycol is adhered to the surface to be modified, for example, which assumes the corresponding existence of functional groups to the surfaces [U.S. Pat. No. 5,908,828]. In these developments, these functional groups must first be generated in some cases by chemical reactions. This is an additional process step and undesirably increases the expense for application of this process.

The anchoring of special peptide sequences on ceramics, polyhydroxy ethyl methacrylate and polyethylene terephthalate is described in U.S. Pat. No. 5,330,911. The process assumes the existence of functional groups and is not suitable for the suppression of non-specific adsorption.

U.S. Pat. No. 5,308,641 discloses a further process is based on polyalkylimine as spacer between the polymer surface and the substance to be adhered. The process has the same disadvantages as described in U.S. Pat. No. 5,330,911 and assumes the existence of corresponding functional groups on the polymer surface.

U.S. Pat. No. 5,897,955 and WO 97/46267 A1 disclose a process wherein the surface of the polymer to be modified is firstly coated with a surfactant, which then only after cross-linking forms the actual surface onto which the substances can be bound. The resulting disadvantage here is also that no adequate masking of the surface is achieved and non-specific adsorption cannot be suppressed.

To increase the compatibility of polymer surfaces, it has been suggested that asymmetric molecules should be bonded onto these surfaces via radical mechanisms. This procedure is therefore bound to specific materials which firstly adsorb on the polymer surface and can then be cross-linked.

According to the U.S. Pat. No. 5,263,992, the surface of biomaterials is firstly covered with a binding molecule in a radical reaction, in which case the binding molecule carries a functional group, onto which surface-modifying substances are bonded. The disadvantage of the process is again that the adsorption of undesirable substances is not suppressed by this structure.

U.S. Pat. No. 5,320,840 describes a polymer which is water-soluble and does not therefore meet the requirements for a solid water-insoluble biodegradable matrix. Many processes such as the one described in U.S. Pat. No. 5,240,747, for example, require drastic conditions for the modification of polymer surfaces, e.g. such as radiation with uv light or the presence of functional groups in the form of amino groups or polyamines (U.S. Pat. No. 5,399,665 and U.S. Pat. No. 5,049,403).

EP 0 844 269 discloses a block polymer with functional groups at both ends, wherein the block polymer is composed from hydrophobic and hydrophilic blocks. The hydrophilic blocks in this case carry as functional groups amino, carboxyl or mercapto groups, which have to be firstly activated for a covalent linkage of surface-modifying molecules of interest, which generally have amino, mercapto, hydroxyl groups or double bonds as functional groups.

WO 95/03356 discloses non-linear block copolymers which are composed from a multifunctional polymer, to which hydrophilic and hydrophobic polymers are bonded. In this case a possible covalent bonding of modifying substances is likewise achieved via a terminal hydroxyl group of the hydrophilic block, e.g. of polyethylene glycol, which requires previous activation.

### SUMMARY OF THE INVENTION

The examples outlined above show the need for biodegradable polymers which have the following properties:

1. Adequate masking of the polymer surface for the suppression of non-specific adsorption of substances;

2. Suppression of non-specific adhesion of living cells;

3. Full biodegradability and biocompatibility of the degradation products;

4. Adjustability of the concentration of functional groups on the polymer surface, which are suitable for the chemical reactions with a plurality of surface-modifying substances;

5. Provision of the possibility of coating the polymer surface with several different substances;

6. To permit binding of the surface-modifying substances before or after processing to shaped bodies (e.g. films, porous sponges, microparticles, nanoparticles, micelles etc.); and

7. Formation of patterns by binding surface-modifying substances on the polymer surface.

Two preconditions must be met in order to permanently anchor surface-modifying substances on polymer surfaces:

1. On their surface the polymers must carry functional groups to which the substances may be chemically bonded.

2. The functional groups must be readily accessible for these chemical reactions.

While known biodegradable polymers such as poly(α-hydroxyesters) [e.g. poly(lactide), poly(lactide-co-glycolide)], polyanhydrides or poly(β-hydroxyesters) have suitable functional groups at both molecule ends, these groups are only accessed on the surface with difficulty. Poly(lactide), for example, has an alcohol and a carboxylic acid function as end group which do not, however, permit binding to the polymer surface for the reasons given above.

To achieve the aforementioned objects, a block copolymer is provided according to the disclosure containing

a hydrophobic biodegradable polymer a),

a hydrophilic biocompatible polymer b),

at least one reactive group c) for covalent binding of a surface-modifying substance d) to the hydrophilic polymer b),

wherein the at least one reactive group c) is an at least bifunctional molecule with at least one free functional group.

According to a further aspect, the disclosure relates to a surface-modified block copolymer which has as additional component a surface-modifying substance d) bonded by means of the reactive group c) as binding link, and a process for the production thereof.

In a preferred configuration, the block copolymers are present as shaped bodies.

The disclosure further relates to the application of the block copolymers in particular in the field of drug delivery, drug targeting, and preferably for tissue engineering.

According to a further aspect the disclosure relates to a process for the production of a block copolymer, wherein the binding of the at least one substance d) to the surface of the block copolymer is achieved by generating a substrate pattern, and the reactive group c) is selected from 1) an at least bifunctional molecule with at least one free functional group and/or 2) a functional group, and block copolymers obtainable with this.

Because of their structure comprising a hydrophobic and a hydrophilic component, the block copolymers according to the disclosure have a surfactant-like character. This causes the polymer, e.g. upon contact with an aqueous medium, to be subject to an orientation wherein the hydrophilic component b) is present in enriched form on the polymer surface, and thus allows free accessibility of surface-modifying substances d) to the reactive group c) for binding.

Therefore, the disclosure relates to polymers, in which a part of the chain, the hydrophilic component b), projects out of the polymer surface and ensures an adequate distance between the polymer surface and reactive group c), as a result of which the binding of surface-modifying substances to the reactive group c) is facilitated.

As a result, special surfaces may be constructed by simple means and prepared for such applications in the best possible way in which the surface of materials serves to assume a specific functionality.

At the same time, the block copolymers according to the disclosure ensure suppression of the non-specific adsorption of molecules and adhesion of cells to their surface.

An important property of the block copolymers described here is the full biocompatibility of the molecule parts used, in which case at least the hydrophobic component a) is biologically degradable.

These polymers also have an advantage in this respect in comparison to systems already described for the modification of surfaces which make use of polystyrene, glass or metals, for example. [Mikulec, L. J. and Puleo, D. A. J. Biomed. Mater. Res. 32 (1996) 203-208; Puleo, D. A. J. Biomed. Mater. Res. 29 (1995) 951-957; Puleo, D. A. Biomaterials 17 (1996) 217-222; Puleo, D. A. J. Biomed. Mater. Res. 37 (1997) 222-228).

In contrast to the named materials, after implantation into the human or animal body, the block copolymers according to the disclosure have the potential to degrade in a specific period of time, depending on the requirement, and to leave the body.

The material properties of the block copolymer can be fixed by the selection of components a) and b) of the block copolymer, i.e. the type and length of the hydrophobic and the hydrophilic polymer chain. For example, the mobility of the fixed substance d) can be varied via the length or structure of the hydrophilic component b). The degradation properties, the mechanical strength and the solubility, for example, in water or an organic solvent of the copolymer can be controlled via the length and structure of the hydrophobic component a).

Hence, by changing the biodegradable lipophilic chain of component a) of the block copolymer, it is possible to increase the period of degradation and increase the mechanical strength of the polymers.

The configuration as block copolymer according to the disclosure supports the orientation, wherein the hydrophilic component predominantly comes to lie on the polymer surface and, for example, promotes the formation of micelles in the aqueous medium.

### BRIEF DESCRIPTION OF THE DRAWINGS

In the drawings:

FIG. 1 shows the binding of a surface-modifying substance d) onto the surface of a block copolymer according to the disclosure via the reactive group c);

FIG. 2 shows the structure of a block copolymer according to the disclosure;

FIG. 3 shows a surface of a block copolymer according to the disclosure coated with different substances d);

FIG. 4 shows images taken by scanning microscope of block copolymers according to the disclosure containing different amounts of polyethylene glycol with a molecular weight of 5000 Da and a reference polymer with no PEG;

FIG. 5 shows ESCA spectra of protein adsorption on different polymer films;

FIG. 6 shows ESCA spectra of peptide adsorption on different polymer films;

FIG. 7 shows images taken by optical microscope of pre-adipocytes 3T3-L1 on different polymer films;

FIG. 8 shows REM images of mesenchymal stem cells from rats on different polymers;

FIG. 9 shows determination of the activity of a block copolymer according to the disclosure via the binding of EDANS; and

FIG. 10 shows the binding of trypsin to a polymer according to the disclosure.

### DETAILED DESCRIPTION OF THE PREFERRED EMBODIMENTS

The subscript indexes used in the polymer designations in the FIGS. relate to the molecular weight (Mn) expressed in kDa.

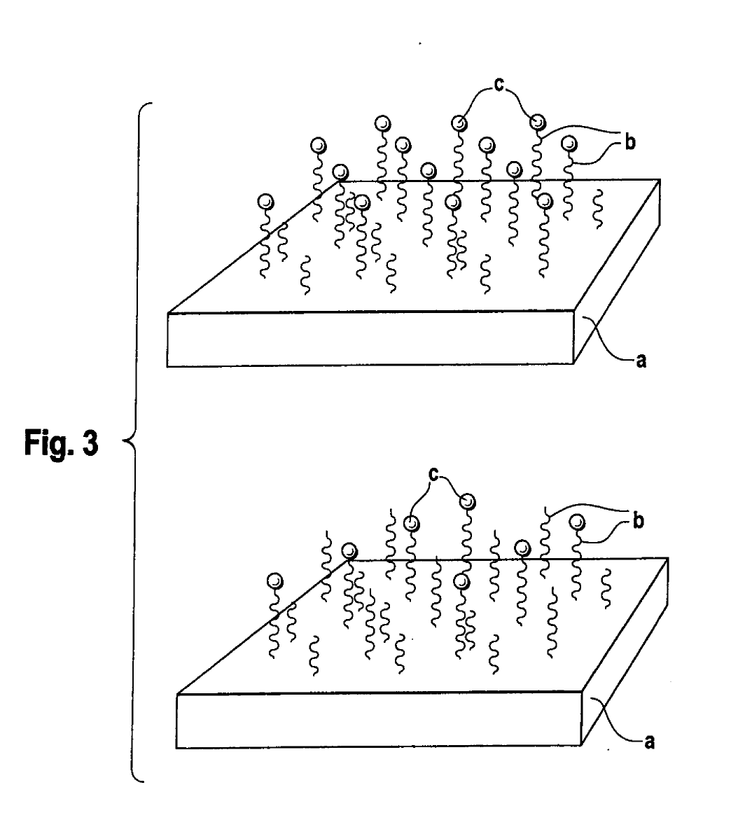

FIG. 2 shows a surface-modified block copolymer according to the disclosure with its essential structural elements, hydrophobic component a), hydrophilic component b) and reactive group c) as well as surface-modifying substance d).

In this case, the hydrophobic component a) serves as carrier and for fixing the entire block copolymer, the hydrophilic component b) serves to make available the reactive group c) for the covalent binding of a surface-modifying substance d) and for masking the surface, and the reactive group c) serves as binding link for the permanent binding of the surface-modifying substance d).

The block copolymer according to the disclosure can be brought into any desired suitable shape for the respective applications, the shaped bodies obtained in this case likewise being subject of the disclosure.

The block copolymer can, for example, be provided as a film, particle in the desired size, e.g. nano- or micro-particle, or three-dimensional shaped body, e.g. monolith. The shaped bodies can be porous. According to a preferred embodiment, the block copolymer forms a porous shaped body in the manner of a sponge, for example.

It is advantageous according to the disclosure that the block copolymer or the shaped bodies formed therefrom are suitable for “instant reactions” with the substance d), which means that they can be produced in advance as stock and stored without problem until application without having to be freshly prepared first for the scheduled application in a time-consuming manner.

The block copolymer can be composed from one or more, also different, blocks comprising the hydrophobic a) and hydrophilic component b), in which case the individual blocks can contain the same monomers possibly with different chain lengths, or different monomers.

According to a preferred configuration, a diblock copolymer is used as block copolymer.

Components a) and b), simultaneously or independently of one another, can be linear or branched, comb- or star-shaped.

Component c) can also be a cross-linked compound, if required.

The surface of the block copolymer can be coated with a single substance or different substances d), the at least one substance d) can form any desired pattern on the surface, e.g. the concentration of the at least one substance d) can be locally constant or variable, it can form a gradient etc.

The type of coating of the surface can be selected in accordance with the application case. Hence, it has been shown that a gradual coating with growth factors can be advantageous.

Any biodegradable hydrophobic polymer known for the named applications can be used as biodegradable hydrophobic component a), like those which have already been specified above. Further polymers can be derived from the literature.

The polymer for component b) can be of synthetic, part-synthetic or natural origin.

They can be poly(a-hydroxyesters, e.g. polylactic acid, polyglycolic acid and their copolymers), poly(e-caprolactam), poly(b-hydroxyesters, e.g. poly(b-hydroxybutyrate) and/or poly(b-hydroxy valerate)), poly(dioxanon), polymalic acid, polytartaric acid, polyorthoester, polycarbonate, polyamide, polyanhydride, polyphosphazene, peptide, polysaccharide, protein and other polymers such as those described in Göpferich A. “Mechanism of Polymer Degradation and Elimination” in: Domb A, Kost J, Wiseman D, eds. Handbook of Biodegradable Polymers. Harwood acad. publ. Inc., 1997: 451-472; Göpferich A: “Mechanisms of Polymer Degradation and Erosion” Biomaterials 17 1996a pp. 103-114 and Göpferich A: Biomaterials 17 (1996a) 103-114; Göpferich A., Eur. J. Pharm. Biopharm. 42 (1996b) 1-11; Leenslag, J. W. et al Biomaterials (1987) 311-314; Park, K et al. Biodegradable Hydrogels for Drug Delivery (1993); Suggs, L. J. and Mikos, A. G. (1996) 616-624.

Further suitable compounds are described, for example, in the Handbook of Biodegradable Polymers (1997) 451-472.

The hydrophobic polymer a) is preferably at least one polymer selected from a polyester, poly-e-caprolactam, poly-a-hydroxyester, poly-b-hydroxyester, polyanhydride, polyamide, polyphosphazene, polydioxanon, polymalic acid, polytartaric acid, polyorthoester, polycarbonate, polysaccharide, peptide and protein.

The hydrophobic polymer a) is, in particular, at least one polymer selected from polylactide, polyglycolide, poly(lactide-co-glycolide), poly-b-hydroxybutyrate and poly-b-hydroxyvalerate.

The hydrophobic component a) is preferably water-insoluble.

The polymers particularly suited as biodegradable component a) are those in which the polymer chain degradation can be brought about by hydrolysis, enzymatic, photolytic or other reactions.

The minimum chain length n measured in monomers amounts to n=2, the upper limit results from the maximum achievable molar masses for the respective monomer in the polymerisation reaction or from the desired properties for the polymer, i.e. depending on the intended application.

As part of the present disclosure the details concerning the molar masses (molecular weight), unless specified otherwise, relate to the numerical mean Mn.

Hence, the chain length of the polymers for component a) can move from few to several thousand monomer units and the polymer can have a molecular weight of over 10 million Dalton.

For example, for polylactide an upper limit of the molar mass of up to 100 000 Da is preferred.

As already mentioned above, the length of the hydrophobic component a) determines the properties of the block copolymer such as the degradation properties and the mechanical strength.

For example, in the case of a combination preferred according to the disclosure of poly(D,L-lactide) (PLA) as hydrophobic component a) and poly(ethylene glycol) (PEG) for the hydrophilic component b), a chain length of the hydrophobic component a) of approx. n<20 leads to water-soluble products. If the PEG content is greater than the PLA content, then water-soluble products can likewise be expected.

A synthetic, part-synthetic or natural biocompatible hydrophilic polymer, which can also be biologically degradable, may be used as hydrophilic component b).

It is built up from at least bifunctional and preferably water-soluble structural elements.

Examples of suitable polymers are polyethylene glycols, polyacrylamides, polyvinyl alcohol, polysaccharides (e.g. modified celluloses and starches), alginates, peptides and proteins.

Preferred examples for the hydrophilic component b) are polyethylene glycol, polypropylene glycol, polyethylene glycol/polypropylene glycol copolymer, polyethylene glycol/polypropylene glycol/polyethylene glycol copolymer, polybutylene glycol, polyacrylamide, polyvinyl alcohol, polysaccharide, peptide and protein.

If a symmetric molecule such as PEG, for example, with two like functional end groups, in this case hydroxyl, is used as hydrophilic component b), it should be ensured during linkage with the hydrophobic component a) that the hydrophobic component does not react with both functional end groups simultaneously, and thus none of the functional end groups remains available as reactive group c) for the covalent binding of surface-modifying substances.

To avoid this problem, a hydrophilic component b) with two different functional end groups is used for the synthesis, as will be explained below by the example of the preferably used PEG, in which case these explanations apply analogously for other symmetric molecules which may be used as hydrophilic component b) for the block copolymer according to the disclosure. Thus, in the case of PEG with two hydroxyl groups as end groups, one of the hydroxyl groups is replaced by another functional group.

For example, poly(ethylene glycol) amine (PEG-NH2) may be used, in which case an end hydroxyl group is replaced by a primary amino group.

This permits the adhesion of the monomers of the hydrophobic component a) to be controlled as part of the synthesis in such a way that the chemical reaction only proceeds at one molecule end.

The type of functional end groups is not restricted in this case to hydroxyl groups and amino groups. Alternatively, thiol groups, double bonds or carbonyl functions may be used for synthesis. Further functional groups are known per se and can be derived from the literature.

The chain length of the hydrophilic component is also determined in accordance with the application and requirement.

For example, the minimum chain length for PEG or of an asymmetric substituted PEG such as PEG-NH2, for example, is at an ethylene unit (ethanolamine).

The upper limit can be set for specific applications in human and animal bodies by the requirement that the released fragments should still be capable of passing through the kidneys and can be excreted.

Suitable molar masses preferably lie at 200 to 10,000 Da, particularly preferred at 1,000 to 10,000 Da, in which case, in particular for applications outside a human or animal body, polymers with higher molar masses of up to several million Da may also be used.

Above all, PEG has proved to be particularly suitable to masking a polymer surface against the adsorption of molecules and the adhesion of cells.

Block copolymers composed from the following combinations are particularly preferred according to the disclosure.

The hydrophobic polymer a) is at least one selected from polylactide, polyglycolide, poly(lactide-co-glycolide). Particularly preferred is a polylactide, e.g. a poly(D,L-lactide), preferably with a molar mass in a range from 1,000 to 100,000, in particular up to 50,000 Da.

The hydrophilic polymer b) is a polyethylene glycol (PEG), wherein polyethylene glycols with a molar mass in a range from 200 to 10,000 Da, in particular 1,000 to 10,000 Da, are particularly preferred.

In principle, the reactive group c) can be any desired functional group or an at least bifunctional molecule, which can form a covalent bond with the selected surface-modifying substance d), with the provision that an at least bifunctional molecule is used as reactive group c) for a block copolymer according to one of claims 1 to 19.

The reactive group c) can comprise:

a single functional group (e.g. amino group, carboxyl group) and thus direct activation of the hydrophilic polymer (e.g. activated acid function or epoxide);

physiological dicarboxylic acids (succinic acid, tartaric acid and variants thereof such as those described in Anderson, G. W. et al. J. Am. Chem. Soc. 86 (1964) 1839-1842), which are provided with terminal groups (succinimidyl esters) in order to achieve the formation of one or two acid amide groupings;

dialdehydes (e.g. glutaric dialdehyde);

special “molecules” for the selective binding of thiols such as those described in Hermanson, G. T. Bioconjugate Techniques (1996), e.g. N-succinimidyl-3-(2-pyridyidithio)propionate (SPDP) or succinimidyl-4-(N-maleimidomethyl)-cyclohexane-1-carboxylate (SMCC);

photoreactive crosslinkers such as those described in Hermanson, G. T. Bioconjugate Techniques (1996), e.g. N-hydroxysuccinimidyl-4-acidosalicylic acid (NHS-ASA), sulphosuccinimidyl-2-(p-acidosalicylic amido)ethyl-1,3′-dithiopropionate (SASD);

splittable crosslinkers such as those described in Hermanson, G. T. Bioconjugate Techniques (1996), e.g. compounds from the above-mentioned groups, which may be split by special reagents e.g. disulphides by hydrogenolysis or by disulphide exchange, glycol groups with periodate (e.g. in the case of tartaric acid), ester groups with hydroxylamine; and

enzymatically splittable molecules such as corresponding peptides, e.g. the sequence Leu-Gly-Pro-Ala, which can be split from collagenase, or oligosaccarides.

Particularly preferred examples of reactive groups c) are those selected from at least one amino group, hydroxyl group, thiol, carboxylic acid, acid chloride, keto group—and in particular for the subject of claims 1 to 19—dicarboxylic acid amide, 3-maleic imidopropionic acid-N-succinimidyl ester and succinimidyl ester.

In principle, the synthesis of the block copolymer according to the disclosure may be achieved in various ways, in which case conventional methods of polymer chemistry are used.

On the one hand, the blocks a) and b) can be synthesized separately and subsequently bonded covalently. Alternatively thereto, it is possible to present a polymer chain and synthesis the missing chain by polymerisation at a polymer chain end. Hence, it is possible, for example, to synthesize block copolymers from poly(D,L-lactide) and poly(ethylene glycol) amine (PLA-PEG-NH2) by presenting PEG-NH2 and synthesizing the biodegradable PLA chain by ring-opening polymerisation from dilactide on the hydroxy end of the PEG-NH2. In principle, the reverse procedure is also possible.

In this case, the reactive group c) can already be present in the polymer obtained, as in the above example, or a functional group present in the hydrophilic component b) can be converted or introduced, where needed, for binding the desired surface-modifying substance d) to a suitable reactive group c).

Hence, the block copolymer can be modified with nucleophilic groups by coupling an at least bifunctional molecule, e.g. disuccinimidyl succinate, to a free end group of component b).

In the simplest case, this reaction can take place in solution, DMSO, for example, is suitable as solvent in the case of PLA-NH2. After preparation of the block copolymer, e.g. to form a suitable shaped body, the reaction can also take place on the surface thereof.

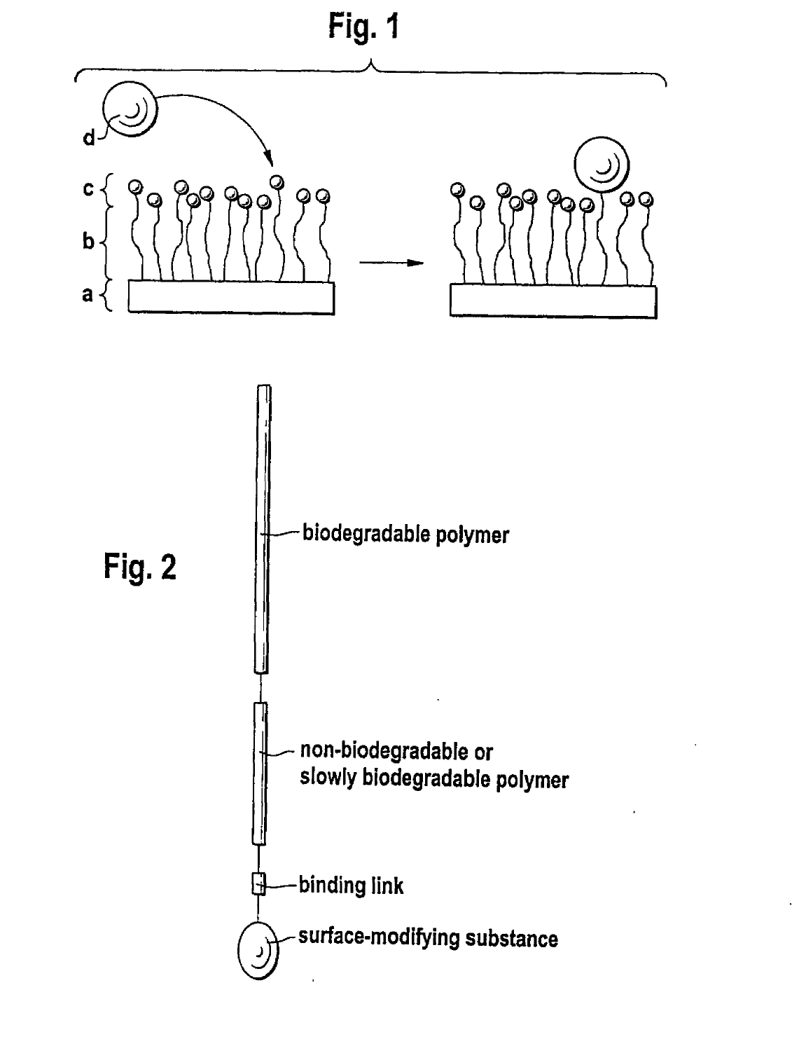

The advantage of activation with a reactive group c) is that the linking of many surface-modifying substances d) proceeds in water. As a result of the reactive group c), which is linked to the hydrophilic block b), this block ends with an active group, which is capable of binding other molecules with nucleophilic functional groups, such as amino groups, for example. FIG. 1 schematically shows the adhesion of a surface-modifying substance to such a polymer surface.

The desired surface property can then be set via the subsequently occurring adhesion of the surface-modifying substance d) to the hydrophilic molecule part b).

Surface-modifying substances d), which may be used for a bond, are generally those carrying a nucleophilic group—e.g. an amino group—, such as carbohydrates, for example, including amongst others: mono-, oligo-, and polysaccharides and glycosides, peptides, proteins, heteroglycans, proteoglycans, glycoproteins, amino acids, fats, phospholipids, glycolipids, lipoproteins, medicinal agents, antibodies, enzymes, DNA/RNA, cells, which can bond directly, for example, via proteins located on the cell membrane, but also dyes and molecular sensors.

Examples for peptides are those with the motif -RGD-, IKVAV or YIGSR and for proteins growth factors, e.g. IGF, EGF, TGF, BMP and basic FGF, proteins and glycoproteins of the extracellular matrix such as fibronectin, collagen, laminin, bone sialo protein and hyaluronic acid. Further substances are described in the relevant literature.

The block copolymer according to the disclosure is particularly suitable for the production of drug targeting systems, drug delivery systems, bioreactors, preferably porous shaped bodies, for therapeutic and diagnostic purposes, for tissue engineering and as emulsifier.

The binding of the surface-modifying substance is explained in more detail below, in general terms and with respect to preferred applications.

For the binding, the block copolymer, like the substance, can be present in solution or the block copolymer forms an immobilized solid surface, to which binds the substance d) present in solution.

In this case, a decisive advantage of the use of the block copolymer according to the disclosure is that under very mild conditions the linking reactions may also be conducted in aqueous medium and therefore sensitive substances d) may also be bonded in.

Hence, proteins can be fixed at room temperature and with a pH suitable for the protein without being denatured on the polymer surface. Alternatively, substances, which are to be bonded to the surface by means of light radiation, can be dissolved in any desired solvent in which the polymer is insoluble. Upon subsequent radiation with uv light, the binding to the surface can then also be linked at room temperature.

Therefore, several conditions are conceivable, in principle, for a binding process, wherein by using the block copolymer according to the disclosure there is sufficient freedom to select optimum conditions with respect to the stability of the substance d) and the polymer.

As a result of the simple type of binding of also unchanged, i.e. non-activated substances d), to the block copolymer with reactive group c) made possible according to the disclosure, the process can be simplified insofar as it is only necessary to dip the finished preshaped polymer carrier, e.g. in the form of micelles, nano-particles, polymer film or polymer sponge, into the solution of substance d) in order to then obtain the finished modified system after a predetermined reaction period (instant reaction).

However, alternatively to the described binding of substance d) to the polymer with reactive group c), the other way round is also possible, namely to first activate the substance d) to be bound with the reactive substance c) for a bond, and then bind the complex comprising substance d) and reactive group c) via the reactive group c) to the component b) of the block copolymer comprising a) and b) to form the finished surface-modified block copolymer according to the disclosure.

However, a disadvantage in this case is that a larger excess of the reactive group c), e.g. a low-molecular dicarboxylic acid here, is generally necessary for activation of the substance d) by binding the reactive group c) in order to prevent the formation of dimers. However, this must be removed again after activation. The consequence of this is, above all with likewise low-molecular substances d), that the purification is more difficult to configure.

In addition to the production of homogeneously coated surfaces, non-homogeneously coated surfaces may also be easily produced with the block copolymers according to the disclosure. This means that, for example, gradients or patterns of the surface-modifying substances d) can also be generated on these polymers. This can be achieved by spot application of the substances d) (e.g. using an ink jet process) or by spot activation of the reactive groups c) by radiation (e.g. with uv light), bombardment with particles, stamping or soft lithography.

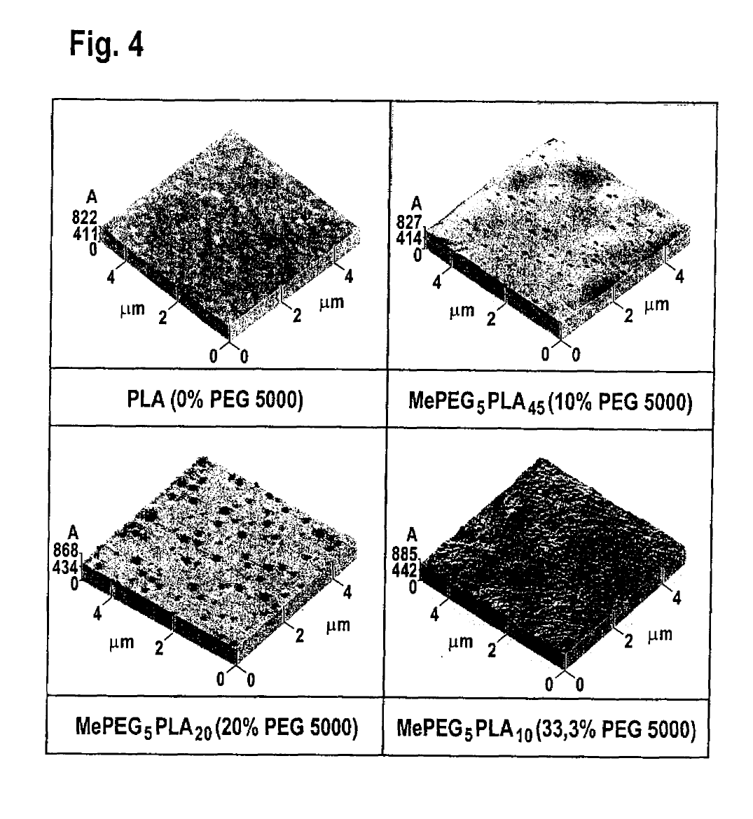

Hence, structured surfaces can be formed which also allow any desired combinations of substances d) to be examined for their effect on cells, for example, or to cultivate combinations of cells in very special spatial orientation to one another or also to construct miniature biotechnological factories using enzymes which perform special reactions in a linked process. FIG. 3 shows such surfaces which are distinguished by two different substances d) and additionally also an inert shorter component.

As part of tissue engineering, it is possible to influence the adhesion, proliferation and differentiation of cells in a better way than previously, since the block copolymers according to the disclosure enable an exact coating of the surface with one or more substances d). At the same time, the non-specific interaction of unwanted substances d), in particular unwanted cells, is suppressed with the polymer surfaces.

As part of drug delivery, it is possible to use the polymers for surface modification, which distributes small polymer particles to specific tissues or organs (drug targeting). This is achieved by binding specific substances d) such as plasma proteins, antibodies or lectins, for example. Further substances d) possible for this are described in the relevant literature.

A further application lies in the chemical bonding of polymers in the form of particles to tissue (bioadhesive systems). An active substance can be distributed in increased concentration to the target tissue by this application.

As a result of the polymer degradation it is to be expected that the substance d) adhered to the polymer block b) is released as part of the hydrolysis. This dynamic process permits the time controlled change of the surface properties of the block copolymer according to the disclosure.

The polymers according to the disclosure may also be used for diagnostic purposes by binding substances d) to their surface, which form a bond with the molecules to be analyzed. The analyzed product can then be separated from the sample together with the polymer (e.g. via a suitable shaped body).

The production of a block copolymer according to the disclosure as well as the subsequent binding of a protein is illustrated below using the example of PEG-PLA to explain the disclosure in more detail.

### WORKING EXAMPLES

### Example 1

### Production of NH₂-PEG-PLA

a) Synthesis of NH2-PEG. Production was conducted in accordance with Yokohama, M. et al. Bioconj. Chem. 3 (1992) 275-276.

The desired amount of ethylene oxide was passed into dry THF in a three-necked flask at −79° C. (dry ice+methanol bath) and dissolved therein. The ethylene oxide bottle was weighed after introduction, and thus the presented amount of ethylene oxide was determined. In accordance with the desired molecular weight of the polymer, the calculated amount of 0.5M solution of potassium-bis-(trimethylsilyl) amide in toluene was then added from a dropping funnel.

The reaction mixture was then stirred in the closed three-necked flask at 20° C. for 36 hours. The polymer solution thus obtained was dropped into the 12-fold amount of ether, and the precipitated polymer was filtered out. After the polymer obtained was dissolved in THF, a small amount of 0.1N hydrochloric acid was added and the silylamide was thus split. The solution of the finished end thus obtained was stirred for 5 minutes at room temperature and once again passed into ether in order to precipitate the pure polymer.

b) Synthesis of NH2-PEG-PLA. Synthesis was conducted in accordance with Kricheldorf, H. R. and Kreiser-Saunders, I. Macromol. Symp. 103 (1996) 85-102; Leenslag, J. W. and Pennings, A. J. Makromol. Chem. 188 (1987) 1809-1814.

The starting products of the synthesis: the NH2-PEG synthesized in accordance with 1a) and cyclic DL-dilactide (3,6-dimethyl-1,4-dioxan-2,5-dion), were each passed into a round flask in the desired weight proportions and dissolved in A.R.toluene. For this, the two flasks were heated at the water separator in order to remove the water still present in the toluene. The solutions thus obtained were than combined in the three-necked flask and once again heated in a permanent nitrogen flow.

The weighed catalyst (tin-2-ethylhexanoate) was then added to the boiling reaction mixture and the mixture was then kept boiling for 8 hours.

The polymer solution thus obtained was passed into a round flask after cooling and rotated three times with dichloromethane in the rotary evaporator until dry. After rotating twice after the addition of acetone, the polymer thus obtained was once again dissolved in acetone and dropped into ice-cooled demineralized water and precipitated thereby. The polymer threads thus obtained were separated through a filter and passed into a vacuum drying cupboard. Determination of the molecular mass can be performed by GPC.

c) Synthesis of the Disuccinimidylester of Tartaric Acid (DSWS). Synthesis was conducted in accordance with Anderson, G. W. et al. J. Am. Chem. Soc. 85 (1964) 1839-1842.

The calculated amounts of tartaric acid and N-hydroxy succinimide were dissolved in a round flask in a mixture comprising dioxan and ethyl acetate (4:1). To this solution the solution of the catalyst (dicyclohexylcarbodiimide) was added in the same solvent mixture and the whole was stirred in an ice bath at 0° C. for 20 hours. The precipitate thus obtained was filtered off and washed with dioxan. The end product was extracted from this precipitate by careful heating with acetonitrile. The solution thus obtained was concentrated to low volume in the rotary evaporator and the product dried in the vacuum cupboard.

d) Synthesis of SWS-NH-PEG-PLA. The starting products obtained in accordance with 1c) and 1b): disuccinimidyl tartaric acid and NH2-PEG-PLA, were dissolved in acetonitrile with a slight excess of the diester and provided with a few drops of triethylamine. After brief heating to boiling, the mixture was stirred for 24 hours. The end product was separated from the acetonitrile by rotation and dissolved in acetone. The polymer solution thus obtained was dropped into water and the precipitate filtered off. The finished active polymer was available after drying in the vacuum.

According to the above-described procedure NH2-PEG-PLA diblock copolymers according to the disclosure were produced with different molecular masses for the components a) and b) for the subsequent experiments or polymers inactivated analogously with methyl groups, in which the reactive group c) was replaced by a methyl group.

### Example 2

### Production of Amino-polyethylene Glycol-poly-L-lactide (NH₂-PEG-PLLA)

The procedure was essentially as in Example 1b). However, cyclic L-dilactide was used instead of the cyclic D,L-dilactide. Further, after rotation three times with dichloromethane, the polymer obtained was once again dissolved in dichloromethane and dropped into ice-cooled diethylether. The polymer thread thus obtained were separated through a filter and passed into a vacuum drying cupboard for drying.

Determination of the molecular weight was achieved by GC and determination of the numerical mean molecular weight was also achieved by 1H-NMR via calculation of the integrals.

### Example 3

### Linkage of Surface-modifying Substances d)

Binding of surface-modifying substances can be conducted in accordance with the processes described in Hill, M. et al. FEBS Lett. 102 (1979) 282-286; Schulman, L. H. et al. Nucleic Acids Res. 9 (1981) 1203-1217.

The linkage of surface-modifying substances d) to the block copolymer according to the disclosure obtained in accordance with Example 1 can occur in two ways, in principle. Firstly, it is possible to bind the substance d) and the block copolymer in solution if the substance d) passes through the subsequent processing steps undamaged. Alternatively, the block copolymer may firstly be processed to the desired form and the substance d) is then linked. In both cases, it should be assured by buffering that an amino group, for example, is present in unprotonated form in order to obtain quantitative yields where possible. Moreover, with buffering the location of the bond to the substance d) can still be controlled if the pH is selected so that only an amino group is present in unprotonated form, for example.

### Example 4

### Characterization of Polymer Films—Properties of the Block Copolymers

4a) Examination of the block copolymers with AFM Scanning microscopy was used to characterize the surface topography of the block copolymers according to the disclosure. For this, the polymers were applied in a 5% solution in chloroform to small square metal plates (5×5 mm) by means of spincasting and then dried. The films thus obtained were then examined with AFM.

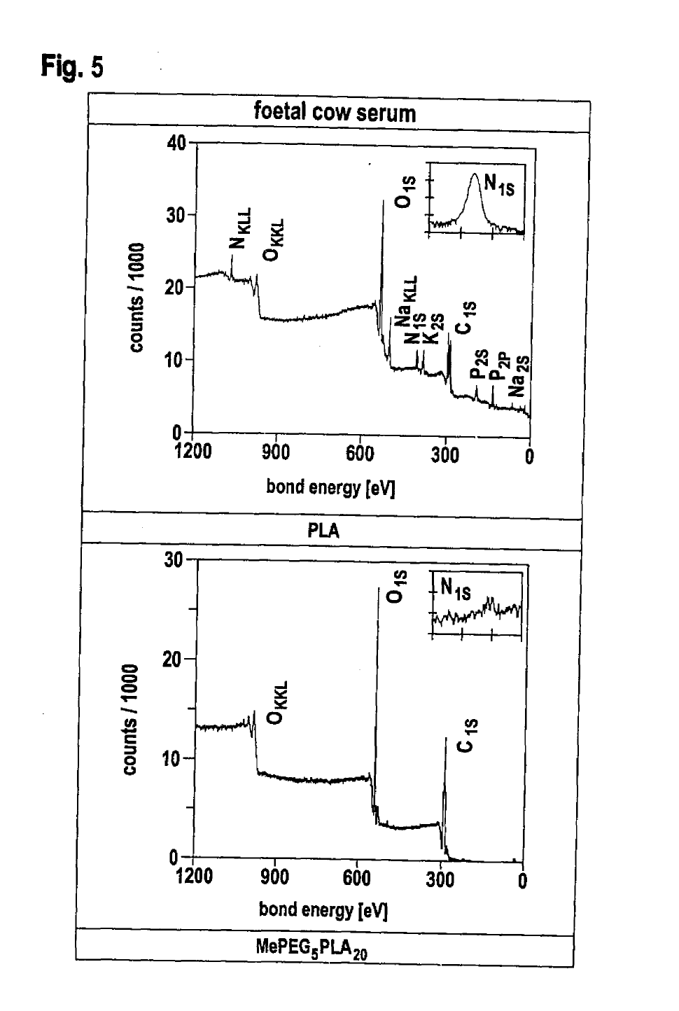

The results are shown in FIG. 4. What are obtained are different concentrations, depending on the polymer examined, of humped raised portions on the polymer surface. The raised portions are crystallites of the polyethylene glycol which increase with the increasing content of polyethylene glycol in the block copolymer. This means that the polymers are distinguished by a phase separation of the blocks and thus an availability of the hydrophilic chains on the polymer surface.

### 4b) Examination of the Protein Adsorption.

Examination of the protein adsorption and its suppression was conducted on different PEG-PLA block copolymers according to the disclosure, which contained a methyl group in place of a reactive group c) and were thus inactivated for the protein bonding.

For examination of the adsorption of proteins onto the polymer films such inactive polymers were poured out onto small metal plates (0.5×0.05 mm) and intensively dried (for at least 2 days in a vacuum), the films thus obtained were then incubated with the protein solutions to be examined and washed off after washing several times with phosphate-buffered (pH=7.4) of isotonic solution. The films thus obtained were then dried again and measured with ESCA.

The model substances were foetal cow serum, atrial natriuretic peptide and salmon calcitonin.

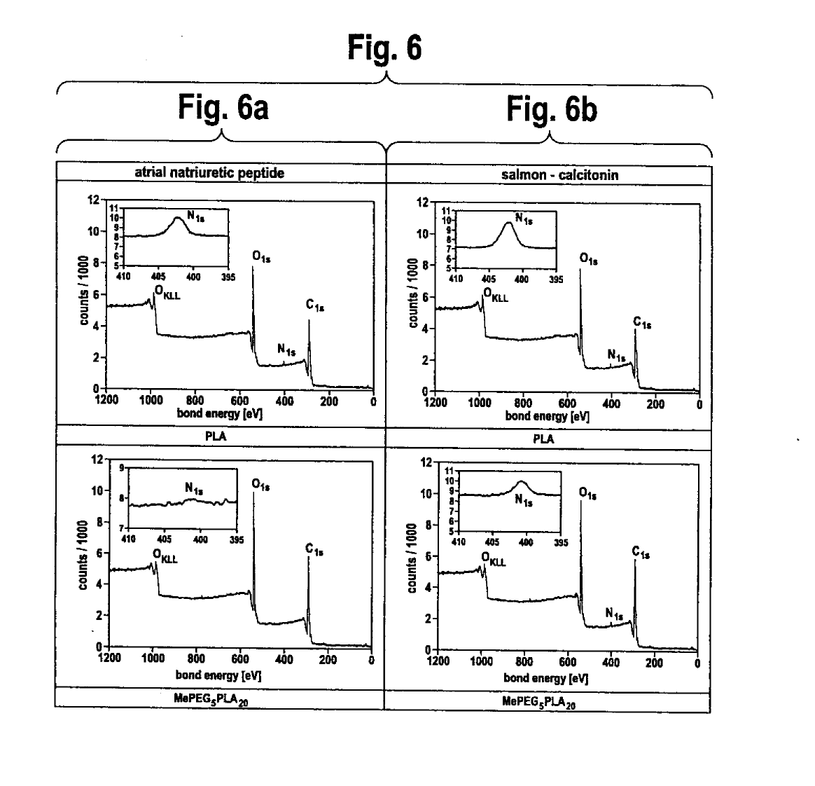

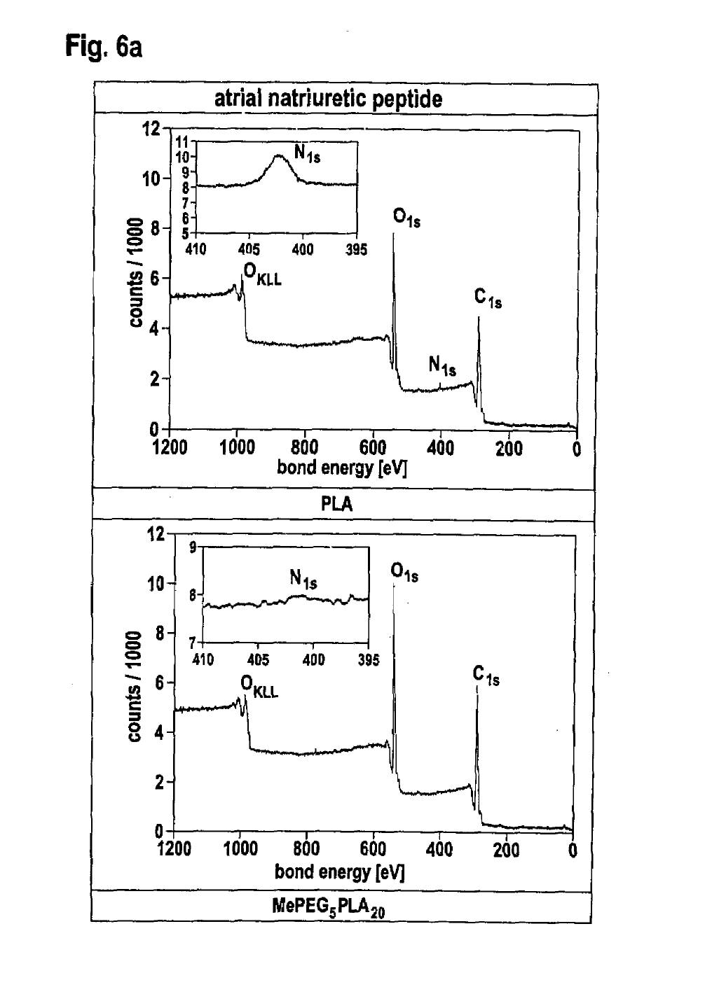

The ESCA spectra served to quantify the adsorbed protein or peptide, since nitrogen was also to be found on the polymer surface as a result of the amino acids of the adsorbed protein. As comparison, polymer films from pure polylactic acid as well as non-incubated polymer films were used. The results are shown in FIGS. 5 and 6.

A suppression of the adsorption dependent on the type of surface-modifying substance d) respectively used was observed. Hence, the adsorption of foetal cow serum was completely suppressed by inclusion of a hydrophilic chain as part of the measurement accuracy (see FIG. 5). In the case of the model peptides calcitonin and atrial natriuretic peptide (ANP), a low adsorption of peptide is still identifiable in part (see FIG. 6).

Therefore, it was established in the result that the block copolymers according to the disclosure are able to control the adsorption of proteins and peptides and can therefore have influence on the behavior of cells which come into contact with the modified polymer surface.

### Example 5

### Examination of the Adhesion Behavior with Respect to Cells

5a) Cells from a pre-adipocyte cell line were put in a suspension on poured films made of different polymers and their adhesion assessed after 5 hours and 24 hours. For this, the suspensions were washed off with buffer prior to microscopy, and thus only the firmly adhered cells were observed.

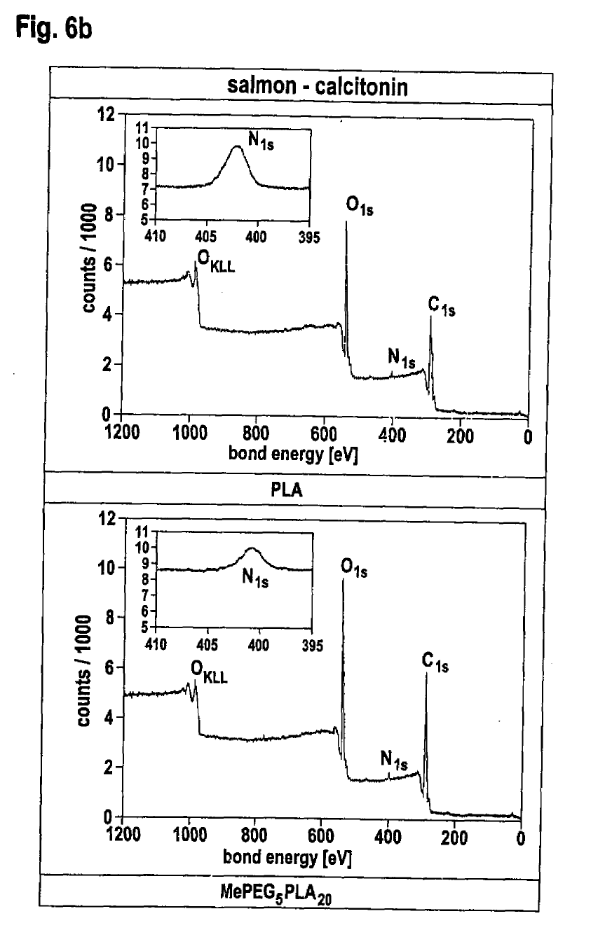

The results are shown in FIG. 7. What is evident are differences in the cell behavior dependent on which polymers were used. Hence, for example, on the MePEG5PLA20 no adhered cells can be recognized both after 5 hours and 24 hours, in which case cells are evident on a small scale on the block copolymer MePEG5PLA20 with the shorter PEG chain, however these adhered only poorly in comparison to the sample composed of lipophilic polylactic acid. After 5 hours only loosely bonded cell aggregates were found and only after 24 hours were single instances of already spread, i.e. firmly bonded, cells found. However, it can be established in the result that the block copolymers according to the disclosure can suppress or reduce the adhesion of cells and can thus prevent or restrict the number of non-specific interactions.

5b) For examination of the adhesion of stem cells of rats, thin polymer films made of different block copolymers according to the disclosure inactivated with methyl (Me-PEG2-PLA20, Me-PEG2-PLA40 and Me-PEG5-PLA45), and for comparison made of PLA, TCPS (tissue culture polystyrene) as well as RG756 (a trade mark for poly(D,L-lactide-co-glycolide 75:25), were poured out on polypropylene discs. The bone marrow stem cells of 6 week old male Sprague Dawley rats with a concentration of 5000 cells per cm3 were cultured onto these films. After 3 hours the morphology of the adhered cells was then observed with the scanning electron microscope.

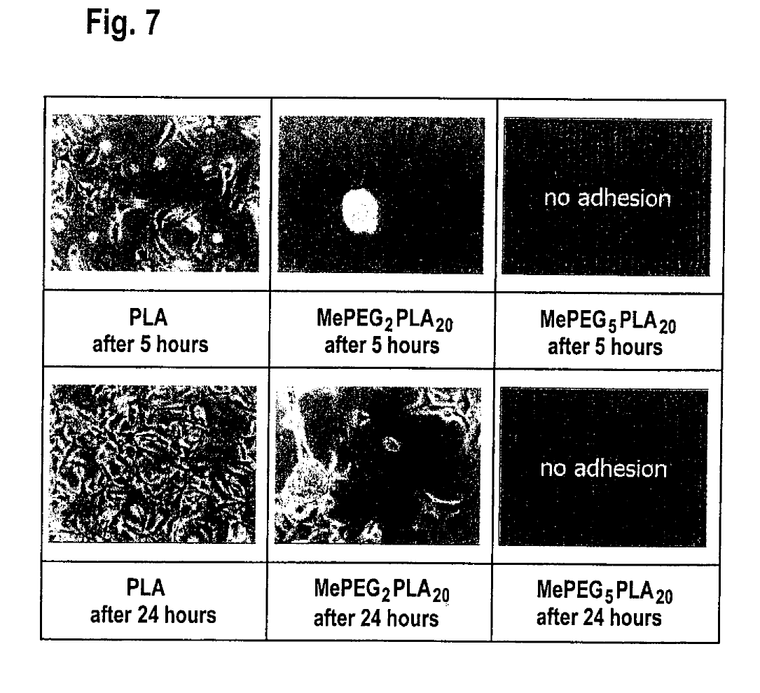

The results obtained are shown in FIG. 8. The number of cells was additionally determined by counting using the optical microscope. It was evident that the number of cells on the block copolymer according to the disclosure was less, the larger the hydrophilic component b) of the polymer. Moreover, the images taken by scanning electron microscope showed that any cells which had adhered to the block copolymer according to the disclosure were in some cases more rounded than on the reference polymers comprising only hydrophobic constituents, which is a clear sign for the low adhesion tendency of the cells to the polymer surface.

### Example 6

### Characterization of the Active Polymers with Respect to their Binding Capabilities

6a) Identification of the Binding Capability with Simple Model Substances with Amino Group in Solution.

For examination of the reactivity in solution, a specific amount of polymer (SWS-NH-PEG2-PLA20) (50 mg) was dissolved in 2000 μl of dimethylformamide (DMF) and mixed with a specific amount of dye (EDANS, 5-((2-aminoethyl)amino)naphthalene-1-sulphonic acid, sodium salt, 0.1-4 mg) which was also dissolved in DMF. In order to exclude any possible protonation of the amino group, 20 μl of triethylamine were added as proton catcher. The solution thus obtained was then incubated overnight in the agitator at 37° C. After the reaction period, 200 μl of the solution were then diluted with 1800 μl of chloroform and the excess precipitated dye was separated by filtration. 200 μl of the clear solutions were then measured by means of gel-permeation chromatography. The amount of covalently bonded dye was determined via the increase in uv absorption at 335 nm.

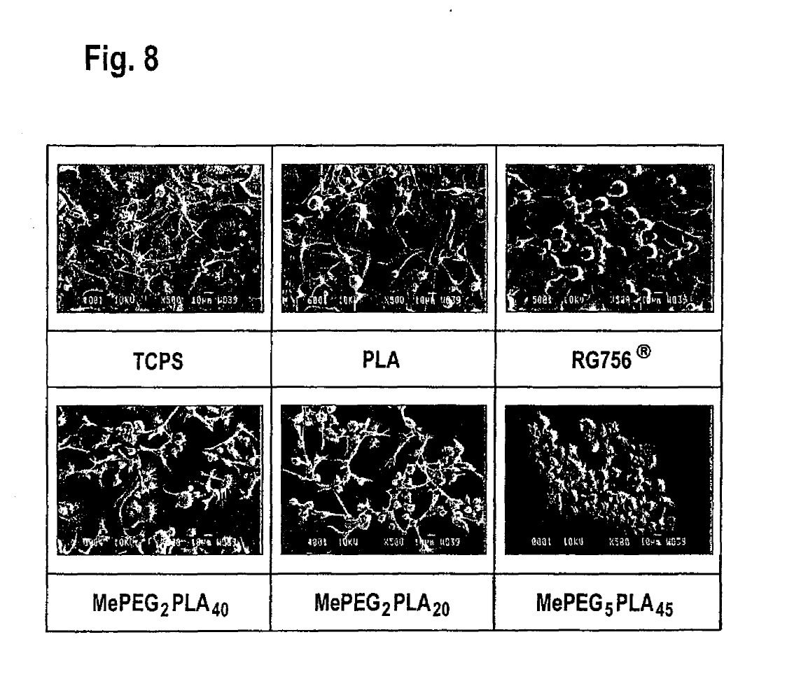

The result is shown in FIG. 9. If the surfaces obtained are evaluated, then a diagram is obtained in which an increase in peak surface may be observed as the amount of dye increases. From a specific amount of dye a plateau is then obtained which is also determined by the restricted number of reactive groups. The amount of reactive groups in a batch of polymer may be simply determined via this determination.

6b) Identification of the Binding Capability with Simple Model Substances with Amino Group on Solid Polymer Surfaces.

The activity on solid surfaces may be examined just as the activity in solution.

For this, films of an active block copolymer according to the disclosure (SWS-NH-PEG2-PLA20), which had been poured onto round glass cover plates, were coated with an aqueous solution of the dye (5-amino eosin) and this solution was then left to work for two hours. The marked films thus obtained were washed with phosphate buffer several times and then dried. The dried films were then dissolved in chloroform and then separated by means of GC possibly adsorbed from covalently bonded dye. The presence of an increased UV absorption was observed with the molecular weight of the polymers. This UV absorption may be explained by a covalent bond between dye and polymer.

### 6c) Binding of Proteins.

For examination of the binding ability also of more complex compounds such as proteins, the enzyme trypsin was used as model substance.

To bind the enzyme to polymer films, films of the various polymers (SWS-NH-PEG2-PLA20 with PLA for comparison) poured onto glass cover plates were incubated with solutions of the enzyme trypsin in phosphate-buffered isotonic common salt solution (PBS buffer). The concentrations of the enzyme used for this amounted to 0.5 or 1.0 mg/ml.

The polymers linked with trypsin thus obtained, after an incubation period of 2 hours, were then washed 3 times with PBS buffer containing 0.05% Tween 20 in order to remove any possibly adsorbed protein as effectively as possible. The films thus washed were then wiped dry and transferred into six-well plates. 2 ml of the reaction medium were then added to each individual well of the plates and the enzymatic reaction was conducted in the incubator for 2 hours at 37° C. The reaction medium was a 1 millimolar solution of benzoyl-L-arginine ethyl ester (BAEE) in tris-buffer with pH=8.0. After 2 hours the enzymatic reaction was stopped by adding an aqueous solution of a trypsin inhibitor composed of soya beans and the transformation of the enzyme substrate was thus terminated. The solutions thus obtained were measured at 253 nm by uv-photometric means.

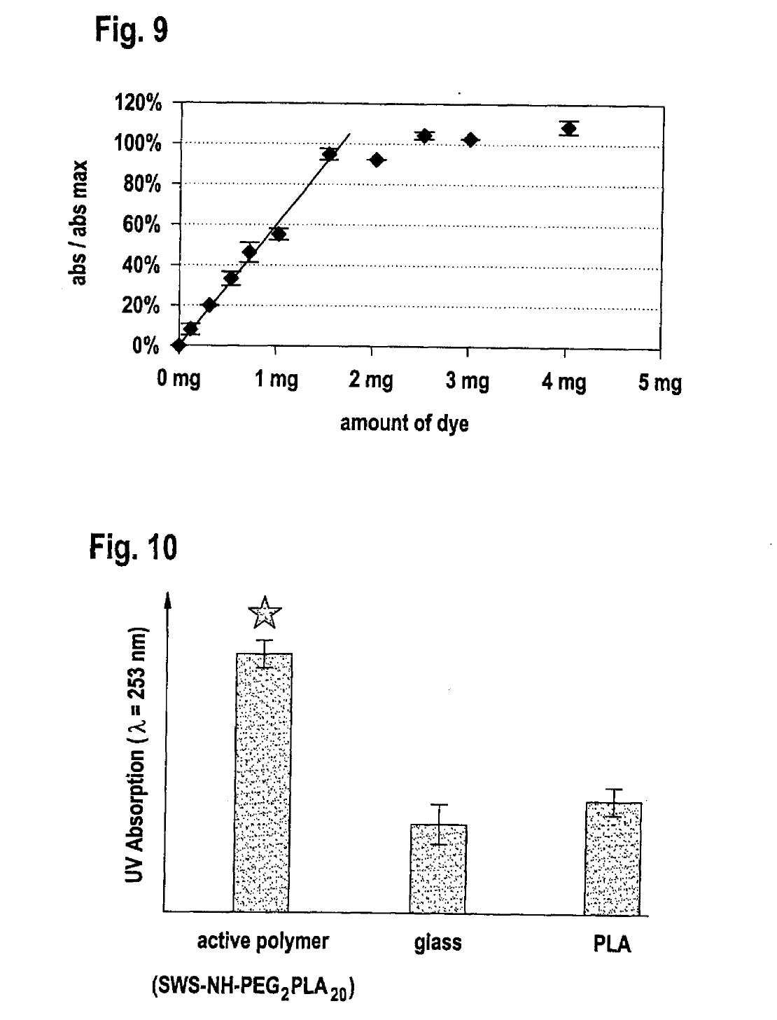

The result is shown in FIG. 10. The comparison with PLA and with the pure glass cover glasses shows a clear increase in the substrate conversion in the case of the block copolymer according to the disclosure which is caused by the amount of covalently bonded enzyme.

1. A composition, comprising a surface coated with a plurality of block co-polymers having an identical orientation, said block co-polymers comprising at least one biodegradable hydrophobic block covalently bound to said surface and at least one hydrophilic block containing a non-carboxyl terminal reactive group suitable for forming a covalent bond with a surface modifying substance in aqueous solution or suspension.

2. The composition of claim 1, wherein said composition is in a form selected from a film, a particle, and a shaped implant.

3. The composition of claim 1, wherein the hydrophobic block comprises a polymer selected from a modified or unmodified polylactic acid, polyglycolic acid, copolymer of lactic acid and glycolic acid, poly(caprolactam), poly(β-hydroxybutyrate), poly(β-hydroxyvalerate), poly(dioxanon), polymalic acid, polytartaric acid, polyorthoester, polycarbonate, polyamide, polyanhydride, polyphosphazene, peptide, polysaccharide, and protein.

4. The composition of claim 1, wherein the hydrophilic block comprises a bifunctional reagent having two different functional end groups.

5. The composition of claim 1, wherein the hydrophilic block comprises a polymer selected from a modified or unmodified polyethylene glycol (PEG), polyvinyl alcohol, polysaccharide, alginate, peptide, and protein.

6. The composition of claim 1, wherein the hydrophilic block comprises a polymer selected from a modified or unmodified polyethylene glycol (PEG), polyvinyl alcohol, polysaccharide, alginate, peptide, and protein.

7. The composition of claim 1, wherein the hydrophobic block comprises a polymer selected from a modified or unmodified polylactic acid, polyglycolic acid, copolymer of lactic acid and glycolic acid, poly(caprolactam), poly(β-hydroxybutyrate), poly(β-hydroxyvalerate), poly(dioxanon), polymalic acid, polytartaric acid, polyorthoester, polycarbonate, polyamide, polyanhydride, polyphosphazene, peptide, polysaccharide, and protein, and the hydrophilic block comprises a polymer selected from a modified or unmodified polyethylene glycol (PEG), polyvinyl alcohol, polysaccharide, alginate, peptide, and protein.

8. The composition of claim 1, wherein the hydrophobic block comprises at least one polymer containing polylactic acid, polyglycolic acid, or a mixture of lactic acid and glycolic acid moieties, and wherein the hydrophilic block comprises at least one polymer comprising a modified or unmodified PEG.

9. The composition of claim 7, wherein said block co-polymers are substantially linear.

10. The composition of claim 1, wherein said block co-polymers are substantially linear.

11. The composition of claim 1, wherein said block co-polymers comprise a diblock co-polymer.

12. The composition of claim 1, wherein said reactive group other than a carboxyl suitable for forming a covalent bond with a surface modifying substance is selected from an amino group, a thiol group, a double bond, an epoxide group, a dicarboxylic acid, and a carbonyl group.

13. The composition of claim 1, wherein said block co-polymers are substantially linear and wherein the hydrophobic block comprises one or more polymers containing polylactic acid, polyglycolic acid, or a mixture of lactic acid and glycolic acid moieties, and the hydrophilic block comprises at least one polymer comprising PEG or a modified PEG having a reactive group other than a carboxyl suitable for forming a covalent bond with a surface modifying substance.

14. The composition of claim 13, wherein said block co-polymers comprise a co-polymer comprising a hydrophobic polylactic acid (PLA) and a hydrophilic PEG modified to have a reactive amino group at a terminus of the block co-polymer.

15. The composition of claim 1, wherein the terminal reactive group comprises a cyclic dicarboxylic acid derivative.

16. The composition of claim 1, further comprising a plurality of surface-modifying substances covalently attached to termini of said block co-polymers.

17. The composition of claim 16, wherein the surface-modifying substance is selected from a cell and a cell-free protein.

18. The composition of claim 1, wherein the plurality of block co-polymers are formed in a pattern.

19. An array of block co-polymers which are covalently attached to a surface having an identical hydrophobic/hydrophilic orientation and which comprise (a) at least one biodegradable hydrophobic block covalently bound to said surface and (b) at least one hydrophilic block containing a terminal reactive group excluding a carboxyl and suitable for forming a covalent bond with a surface modifying substance in aqueous solution or suspension.

20. A plurality of block co-polymers forming a co-polymer array on a polymer surface, having an identical orientation, and comprising:
at least one biodegradable hydrophobic block covalently bound to said surface; and
at least one hydrophilic block containing a terminal reactive group, other than a carboxyl, that is suitable for forming a covalent bond with a surface modifying substance in aqueous solution or suspension.

21. A substantially linear block co-polymer, comprising:
a) a hydrophobic block comprising at least one hydrophobic polymer covalently bound at a proximal end to a surface; and
b) a hydrophilic block comprising at least one hydrophilic polymer covalently bound to the hydrophobic block, wherein a distal end of the hydrophilic block contains a terminal reactive group which excludes a carboxyl;
wherein the reactive group is suitable for forming a covalent bond with a surface modifying substance in aqueous solution or suspension.

22. The block co-polymer of claim 21, wherein:
the hydrophobic block comprises one or more polymers containing polylactic acid, polyglycolic acid, or a mixture of lactic acid and glycolic acid moieties,
the hydrophilic block comprises a modified polyethylene glycol having an amino group, the block co-polymer has a reactive group other than a carboxyl, and
the reactive group is suitable for forming a covalent bond at a distal terminus with a surface modifying substance.

23. The block co-polymer of claim 22, wherein the hydrophobic block comprises polylactic acid and wherein the hydrophilic block comprises poly(ethanolamine) (PEG-NH₂).

24. The block co-polymer of claim 23, wherein the hydrophobic block has a molecular weight of 100-100,000 Da and the hydrophilic block contains from a single ethanolamine to a poly(ethanolamine) having a molecular weight of about 10,000 Da.
